(19) United States
(12) Patent Application Publication
(10) Pub. No.: US 2012/0208715 A1
(43) Pub. Date: Aug. 16, 2012

(54) METHODS AND COMPOSITIONS FOR DIAGNOSIS OF ACUTE MYOCARDIAL INFARCTION (AMI)

(76) Inventors: John T. McDevitt, Houston, TX (US); Craig S. Miller, Nicholasville, KY (US); Jeffrey L. Ebersole, Lexington, KY (US); Nicolaos Christodoulides, Austin, TX (US); Pierre N. Floriano, Missouri City, TX (US)

(21) Appl. No.: 13/390,619

(22) PCT Filed: Aug. 20, 2010
(86) PCT No.: PCT/US10/46134

§ 371 Date: Apr. 30, 2012

Related U.S. Application Data
(60) Provisional application No. 61/235,517, filed on Aug. 20, 2009.

(51) Int. Cl. C40B 30/04; G01N 33/573; G01N 33/566

(52) U.S. Cl. 506/9; 435/7.92; 435/7.4; 436/501

(57) ABSTRACT

Embodiments of the invention utilizes advanced detection methodologies, such as the lab-on-a-chip (LOC) technology, as a cost-effective, efficient, ultra-sensitive rapid method for diagnosing Acute Myocardial Infarction (AMI) in human subjects. In certain aspects, multiple biomarkers of AMI are concurrently detected and measured in serum and saliva to provide a more efficient, sensitive and accurate diagnosis of AMI.

This application claims priority to U.S. Provisional Patent Application Ser. No. 61/235,517 filed Aug. 20, 2009, which is incorporated herein in its entirety.

This invention was made with government support under grant number 9 R01 EB000549-04A1, UO1 DE15017, and UO1 DE017793 awarded by the National Institute of Health. Consequently, the government has certain rights in the invention.

### BACKGROUND OF THE INVENTION

I. Field of the Invention

The present invention relates generally to the fields of medicine, physiology, diagnostics, and biochemistry. In certain embodiments, the invention relates to assessment of biomarkers indicative of acute myocardial infarction (AMI).

II. Background

Cardiovascular disease (CVD) is the leading cause of death in developed countries with enormous health, social, and economical consequences. In the United States alone, the projected cost of CVD in 2005 is estimated at $431.8 billion, including health care services, medications, and lost productivity. Atherosclerotic Heart Disease (ASHD) develops when lipids and inflammatory cells accumulate in the walls of coronary arteries, forming atherosclerotic plaques. As ASHD progresses, clinical manifestations may develop, including the occurrence of angina.

Acute Coronary Syndrome (ACS), which includes unstable angina and acute myocardial infarction (AMI), is associated with plaque rupture and thrombus formation in a coronary vessel, resulting in myocardial ischemia and often necrosis.

According to the American Heart Association (Heart and Disease Statistics—2004), the following dire morbidity and mortality statistics are associated with ASHD in the United States: ASHD is the primary cause of death in America today and was responsible for more than one third of U.S. deaths in 2004. Further, 13.2 million people (7.2 million males and 6.0 million females) living today have experienced a heart attack, angina or both; approximately 330,000 people a year will die of an ACS event inside or outside of the emergency room and 1.2 million Americans are expected to have a new or recurrent coronary event this year. In 2008, an estimated 770,000 Americans will have a new coronary attack, and about 430,000 will have a recurrent attack. It is estimated that an additional 175,000 silent first myocardial infarctions occur each year. About every 26 seconds, an American will have a coronary event, and about every minute someone will die from a coronary event.

A heart attack, known in medicine as an (acute) myocardial infarction (AMI or MI), occurs when the blood supply to part of the heart is interrupted. Heart attacks or AMI are the leading cause of death for both men and women all over the world. The diagnosis of AMI is usually predicated on the World Health Organization (WHO) criteria of chest pain, ECG changes, and increases in biochemical markers of myocardial injury. About half of the patients with “typical” symptoms do not have AMI. Similarly, a significant number of patients that do experience an AMI are sent home misdiagnosed as having a cold. The diagnosis of AMI is particularly difficult in the elderly, where relatively minor symptoms may reflect acute ischemia. The ECG is specific for AMI, provides additional information regarding localization and the extent of the injury but lacks sensitivity. Sometimes, it is not easy to distinguish past injury from a more recent one. Therefore, there is a need to develop a more sensitive, accurate and cost-effective method for diagnosing and timing AMI.

### SUMMARY OF THE INVENTION

Embodiments of the invention include methods for an analysis of a body fluid for establishing a diagnosis or a prognosis of a subject with regard to acute myocardial infarction. In certain aspects, the analysis of the body fluid is for establishing that the subject has or is suffering from acute myocardial infarction or the subject is at risk of suffering acute myocardial infarction. In a further aspect, the acute myocardial infarction is a recurrent cardiac event. In still further aspects, the analysis of the body fluid include measuring a level of two or more biomarkers in a sample from the subject. In yet a further aspect, biomarkers are assessed or evaluated concurrently. In certain aspects, biomarkers are assessed concurrently and on a platform comprising normalization and evaluation controls such as concentration titers of biomarker being measured. In further aspects, one or more biomarkers in a sample may be detected, measured or quantified by a detection device or system, e.g., lab-on-a-chip.

As used herein, biomarkers are substances used as indicators of a biologic state. It has a characteristic that is objectively measured and evaluated as an indicator of normal biologic processes, pathogenic processes, or pharmacologic responses to a therapeutic intervention. In certain aspects, biomarkers are proteins, protein fragments, or polypeptides.

All of this may be done in a non-invasive fashion at the point-of-care using saliva and lab on a chip (LOC) technology. Lab on a chip technology as well as point of care apparatus and sampling methodology can be found in various PCT publications, each of which are incorporated herein by reference in their entirety and include WO 2005/059551, WO 2007/002480, WO 2001/055702, WO 2007/005666, WO 2005/085855, WO 2003/090605, WO 2005/085854, WO 2005/090983, WO 2005/083423, WO 2000/004372, WO 2001/006253, WO 2001/006244, WO 2001/006239, WO 2001/055952, WO 2001/055701, WO 2001/055703, WO 2001/055704, WO 2002/061392, WO 2004/009840, WO 2004/072097, WO 2004/072613, WO 2005/085796, WO 2007/134191, and WO 2007/134189.

Certain embodiments include methods for evaluating a subject suspected of having suffered an acute myocardial infarction comprising simultaneously measuring levels of C-reactive protein (CRP), myoglobin and myeloperoxidase (MPO) in a saliva sample obtained from a subject suspected of suffering an acute myocardial infarction. The method can further comprise measuring the levels of one or more additional marker such as cardiac troponin I (cTnI), MMP-9, IL-6, IL1β, soluble Vascular Cellular Adhesion Molecule-1 (sVCAM-1), fractalkine, soluble Intercullular Adhesion Molecule-1 (sICAM-1), B-natriuretic peptide (BNP), creatine kinase-MB (CK-MB), or E-Selectin in the saliva sample. In certain aspects, the marker levels are measured by a microfluidic sensor array, such as a lab-on-a-chip (LOC) sensor.

In certain aspects a patient is identified as be suspected of having or as had an acute myocardial infarction, for example the patient can be experiencing chest pains, etc.

In further aspects the method of measure the biomarkers is completed (i.e., measurement of levels obtained) in less that 10, 20, 30, 40, 50, 60 minutes of obtaining a sample from a subject. In certain aspects the measurement is complete in less than an hour after obtaining the sample.

In further embodiments the methods include simultaneously measuring the levels of CRP and one or more of MMP-9, IL1β, slCAM-1, or MPO.

In further embodiments the methods include simultaneously measuring the levels of CRP and one or more of MMP-9, IL1β, slCAM-1, MPO, adiponectin, MCP-1, or Gro-α.

In further embodiments the methods include simultaneously measuring the levels of CRP and one or more of MMP-9, IL1β, slCAM-1, MPO, adiponectin, MCP-1, Gro-α, E-selectin, IL-18, ENA-78, or sVCAM-1.

In further embodiments the methods include simultaneously measuring the levels of CRP and one or more of MMP-9, IL1β, slCAM-1, MPO, adiponectin, MCP-1, Gro-α, E-selectin, IL-18, ENA-78, sVCAM-1, MYO, CK-MB, TnI, or BNP.

In further embodiments the methods include simultaneously measuring the levels of CRP, MMP-9, IL1β, slCAM-1, MPO, adiponectin, MCP-1, Gro-α, E-selectin, IL-18, ENA-78, sVCAM-1, MYO, CK-MB, TnI, BNP, fractalkine, rantes, IL-6, sCD40-L, and TNF-α.

Certain embodiments are directed to methods for measuring the level of the biomarkers. These methods include, but are not limited to, a microfluidic sensor array, an immunoassay test, μ-array measurement, a proteomic array, or Luminex®. In certain aspects, the microfluidic sensor assay is the LOC technology referenced above. In further aspects, the immunoassay test is an ELISA.

In a further embodiment, the threshold level for a biomarker may indicate the presence or absence of a biomarker, or indicate a risk level division in which the measured biomarker level falls. In certain aspects, the threshold level can be determined by the steps of: (a) obtaining a sample from each of a plurality of subjects including cardiac healthy subjects and cardiac disease subjects at risk of or having cardiovascular disease; (b) quantifying the level of the biomarkers in each sample; (c) comparing the level between the cardiac healthy subjects and the cardiac disease subjects; (d) identifying and selecting a biomarker that distinguish the cardiac healthy subjects from the cardiac disease subjects; and (e) determining a threshold level for the selected biomarker based on discriminatory concentration for the selected biomarker (e.g., that level that distinguishes between the two groups at a particular relevance).

In a further embodiment, an analysis of a body fluid include, but is not limited to, measuring 1, 2, 3, 4, 5, 6, 7, 8, 9, 10, 15, 20, 25, 30, 35, or 40 biomarkers concurrently or sequentially. Biomarkers include, but are not limited to, LDL, HDL, C-reactive protein (CRP), adiponectin, Apolipoprotein A (ApoA), Apolipoprotein B (Apo B), E-selectin, IL-1α, IL-1β, IL-4, IL-5, IL-6, IL-10, IL-13, IL-18, creatinine kinase-MB (CK-MB), β-natriuretic peptide (BNP), FABP (cardiac fatty acid protein), TNF-α, MCP-1, MMP-9, MPO, Intercellular Adhesion Molecule (ICAM), Vascular Cellular Adhesion Molecule (VCAM), sCD40L, ENA78, fractalkine, PIGF, PAPP-A, RANTES, sCD40L, vWF, D-dimer, IMA, FFAu, Choline, cTnT, Cardiac troponin I (cTnI), Myoglobin, NT-proBNP, MMP or a combination thereof.

In certain aspects the biomarker LDL, HDL, C-reactive protein (CRP), adiponectin, Apolipoprotein A (ApoA), Apolipoprotein B (Apo B), E-selectin, IL-1β, IL-4, IL-5, IL-6, IL-10, IL-13, IL-18, creatinine kinase-MB (CK-MB), β-natriuretic peptide (BNP), FABP (cardiac fatty acid protein), TNF-α, MCP-1, MMP-9, MPO, Intercellular Adhesion Molecule (ICAM), Vascular Cellular Adhesion Molecule (VCAM), sCD40L, ENA78, fractalkine, PIGF, PAPP-A, RANTES, sCD40L, vWF, D-dimer, IMA, FFAu, Choline, cTnT, Cardiac troponin I (cTnI), Myoglobin, NT-proBNP, or MMP can be used in combination with a second or third biomarker selected from LDL, HDL, C-reactive protein (CRP), adiponectin, Apolipoprotein A (ApoA), Apolipoprotein B (Apo B), E-selectin, IL-1α, IL-1β, IL-4, IL-5, IL-6, IL-10, IL-13, IL-18, creatinine kinase-MB (CK-MB), β-natriuretic peptide (BNP), FABP (cardiac fatty acid protein), TNF-α, MCP-1, MMP-9, MPO, Intercellular Adhesion Molecule (ICAM), Vascular Cellular Adhesion Molecule (VCAM), sCD40L, ENA78, fractalkine, PIGF, PAPP-A, RANTES, sCD40L, vWF, D-dimer, IMA, FFAu, Choline, cTnT, Cardiac troponin I (cTnI), Myoglobin, NT-proBNP, or MMP.

In further aspects, the sample may be a body fluid, such as serum, saliva, urine, blood, blood plasma, or cerebrospinal fluid.

Abbreviations include: AMI, acute myocardial infarction; ECG, electrocardiogram; STEMI, ST elevation myocardial infarction; NSTEMI, non-STEMI; MYO, myoglobin; CK-MB, creatine kinase-MB; cTnT, cardiac troponin T; cTnI, cardiac troponin I; POC, point of care; LOC, lab-on-a-chip; CRP, C-reactive protein; UWS, unstimulated whole saliva; BNP, brain natriuretic peptide; IL, interleukin; MCP-1, monocyte chemoattractant protein-1; MPO, myeloperoxidase; sCD40L, soluble cluster of differentiation ligand; TNF-α, tumor necrosis factor-α; RANTES, regulated on activation, normal T expressed and secreted; sVCAM-1, soluble vascularization cellular adhesion molecule-1; ENA-78, epithelial cell-derived neutrophil-activating peptide 78; Gro-α, growth related protein-α; sICAM-1, soluble intercellular adhesion molecule-1; MMP-9, matrix metalloprotease-9; AUC, area under the curve.

An example of results using certain aspects of the invention have been published in Floriano et al., Clinical Chemistry, 55:8, 1530-1538 (2009), which is incorporated herein by reference in its entirety.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Biomarker expression and diagnostic accuracy values in different formats are provided. The bar graphs on the left show ratios of median concentrations of biomarkers for the diseased patients over those of healthy controls. Data is provided both in serum (red) and in saliva (blue) with actual ratio values indicated next to each bar. On the right hand side of the illustration, text values for the AUC are provided. Here entries for which p<0.05 are denoted with an asterisk. Included here also are various methods of ranking the various biomarkers: r1 is the rank obtained by each biomarker according to its AUC, r2 is the ranking obtained from the ratio of median diseased over median healthy (up- and down-regulated biomarkers are ranked equally), and r3 ranking results from the p-value related to the statistical significance of the difference between the medians of diseased and control populations. An aggregate ranking (R) is also provided based on the averages of r1, r2, and r3.

FIGS. 2A-2D. Four receiver operating characteristics (ROC) plots generated in an automated fashion are provided to explore the diagnostic accuracy of various models. For the initial model, all variables are included regardless of their statistical or biological significance (“enter”, A). For the second model, only significant variables are entered sequentially into the model (“forward”, B). For the third case, all independent variables are first entered into the model and then removed sequentially if not found significant (“backward”, C). Finally, for the forth model all significant variables are entered sequentially and the model is recalculated if a variable is found to become non-significant and excluded after inclusion of another independent variable (“stepwise”, D). The retained biomarkers are shown for each model along with the AUC and the best average sensitivity and specificity values.

FIGS. 3A-3D. Four receiver operating characteristics (ROC) plots are generated in a manual fashion so as to explore the diagnostic accuracy of various models that include only FDA-approved biomarkers in the context of saliva tests for AMI diagnosis. The following ROC analysis were obtained in this manner: (A) the combined use of CRP, MPO, and MYO; (B) the panel including CRP and MYO; (C) the combined use of CRP, MPO, and MYO with companion ECG; (D) the panel including CRP and MYO with ECG.

FIGS. 4A-4D. A Multiplex lab-on-a-chip (LOC) demonstration for AMI diagnosis is provided. (A) First, a scanning electron micrograph of the silicon microchip is shown with the LOC fluidic compartment on the left. An immuno-schematic depicts the sandwich type immunoassay Clinical Chemistry detection modality and the analyte of interest (here, CRP, IL-1β, MYO, or MPO antigens are represented in blue). In the other panels, examples of fluorescence micrograph of a LOC multiplex assay for CRP, IL-1β, MYO, and MPO are shown for healthy control (B), NSTEMI (C), and STEMI (D) patients.

### DETAILED DESCRIPTION

Cardiac biomarkers hold great promise for diagnosing AMI patients. However, the current technologies used for the measurements of these biomarkers are limited to testing one biomarker at a time using long, expensive and laboratory-based procedures with a detrimentally-slow turnaround of results. Furthermore, individually these biomarkers are unlikely to provide a complete picture of specific cardiac disease processes.

Therefore, the inventors developed a method offering optimal AMI diagnosis based on information from multi-analyte-based screening that can be most informative when applied at “the point-of-care,” such as in the ambulance or emergency room.

Furthermore, even though this method can be applied for serum, which has been the traditional diagnostic fluid for cardiac diagnostics, additional aspects of the disclosure relies in the utility of saliva as a diagnostic or prognostic fluid for AMI. The newly designed cardiac test in saliva is non-invasive in comparison to established AMI testing methods and is naturally amenable to multiplexing through the use of customizable platforms that can contain a chosen number of sensor elements that can be addressed spatially within a nano-biochip sensor device.

### I. ACUTE MYOCARDIAL INFARCTION (AMI) BACKGROUND

Myocardial infarction is a common presentation of ischemic heart disease. The World Health Organization (WHO) estimated that in 2002, 12.6 percent of deaths worldwide were from ischemic heart disease. Ischemic heart disease is the leading cause of death in developed countries, but third to AIDS and lower respiratory infections in developing countries. Thus, a sensitive and accurate prognosis and diagnosis in patients with acute myocardial infarction is vital as provided in the present disclosure.

A. Difference Between Acute Myocardial Infarction (AMI) and Atherosclerosis

Atherosclerosis is a disease affecting arterial blood vessels. It is a chronic inflammatory response in the walls of arteries, in large part due to the accumulation of macrophage white blood cells and promoted by low density (especially small particle) lipoproteins (plasma proteins that carry cholesterol and triglycerides) without adequate removal of fats and cholesterol from the macrophages by functional high density lipoproteins (HDL), (see apoA-1, Milano). It is commonly referred to as a “hardening” or “furring” of the arteries. It is caused by the formation of multiple plaques within the arteries.

Atherosclerosis is the gradual buildup of cholesterol and fibrous tissue in plaques in the wall of arteries (such as the coronary arteries), typically over decades. Blood stream column irregularities visible on angiography reflect artery lumen narrowing as a result of decades of advancing atherosclerosis. Plaques can become unstable, rupture, and additionally promote a thrombus (blood clot) that occludes the artery; this can occur in minutes. When a severe enough plaque rupture occurs in the coronary vasculature, it leads to myocardial infarction (necrosis of downstream myocardium).

In contrast to atherosclerosis as being chronic, slowly progressing and cumulative, acute myocardial infarction is an acute event, usually due to acute thrombotic occlusion of an epicardial vessel, which occurs as a consequence of sudden disruption of the atherosclerotic plaque associated with spontaneous fissuring or rupture, totally occluding the artery and preventing blood flow downstream. Thus, diagnosis of atherosclerosis may not completely apply to that of AMI and a different method of diagnosis of AMI is needed in addition to current methods of diagnosis of atherosclerosis.

B. Present Diagnosis of AMI

Currently, the diagnosis of AMI is usually predicated on the World Health Organization (WHO) criteria of chest pain, electrocardiogram (EKG) changes, and increases in blood levels of markers of myocardial injury. Unfortunately, a significant number of AMI cases are missed or diagnosed late, while about half of the patients with “typical” symptoms do not have AMI.

The diagnosis of AMI is particularly difficult in the elderly, where relatively minor symptoms may reflect acute ischemia. The EKG is specific for AMI, but lacks sensitivity as it misses AMI cases with no ST-elevation, i.e. NSTEMI patients. The EKG also provides additional information regarding localization and the extent of the injury. However, sometimes, it is not easy to distinguish remote injury from a more recent one. In contrast, biochemical markers have excellent sensitivity for diagnosing AMI. By combining the most sensitive and the most specific tests, diagnostic accuracy can be enhanced.

The crucial step in ruling in/out the diagnosis of AMI is the measurement of myocardial enzymes in the serum. The rate of release of specific proteins differs depending on their intracellular location, molecular weight, and the local blood and lymphatic flow. The temporal pattern of marker protein release is obviously of diagnostic importance. Here, delays in patient entry from the onset of infarction may miss elevations of cardiac enzymes that are elevated early from the onset of infarction (e.g., myoglobin) which may affect the diagnosis and translate in delay of treatment (i.e., reperfusion), which ultimately could lead to increased mortality in myocardial infarction.

According to a recent report, emergency rooms are so overwhelmed with patients that it takes nearly an hour for 25% of heart attack victims to be seen by a doctor. During the 1997-to-2004 study period, as the number of emergency room visits rose and the number of emergency departments declined, the time it took for any patient to see a doctor stretched to 36% of the patients. But the increase was, in fact longer, to 40%, for patients identified by a triage nurse as needing help immediately. Surprisingly, the patients who saw the greatest increase in waiting time were ones whose lives most depend upon rapid treatment: those having a heart attack. Every minute of delay in treatment during a heart attack increases the likelihood that the patient will die, but heart attack patients waited 150% longer for care by the end of the study period, or 20 minutes on average. One in four waited 50 minutes or more. Added to that is the time the patient, or the close relative, took to call the emergency in, and the time it took to transport him/her to the ER.

Therefore, there is a need for improvement on minimizing the time delay between arrival at the emergency department and performance of reperfusion, by either pharmacological or catheter-based approaches. Methods that make assessment easier, faster and predictable as disclosed in the present invention could indeed save lives.

### II. UTILITY OF DETECTION METHODS FOR CARDIAC DIAGNOSIS AND RISK ASSESSMENT OF AMI AT THE POINT OF CARE (POC)

Certain embodiments, as applied to AMI diagnosis, utilize a lab-on-the-chip (LOC) microfluidic assay platform to target multiple clinically relevant biomarkers in physiological fluids with reduced sample, reagent, and assay time requirements. In other embodiments, alternative or complimentary advanced detection methodologies (such as proteomic chips, ELISA and Luminex) may be used to target the same and/or other biomarkers of AMI. In these embodiments, this new invention promises to have a significant impact on AMI clinical diagnostics, especially at the near-patient or point-of-care setting.

A. Lab-on-a-Chip (LOC)

Remarkable advances have been made recently in the development of miniaturized sensing and analytical components for use in a variety of biomedical and clinical applications (Liu et al., 2003; Manz et al., 1990; Situma et al., 2005; Tudos et al., 2001; Verpoorte and De Rooij, 2003; Whitesides, 2005). However, the ability to assemble and interface individual components in order to achieve a high level of functionality in complete working devices continues to pose a daunting challenge for the scientific community as a whole. Lessons learned from the microelectronics and computer-software industries provide inspiration for what may be gained from the marriage of microelectronics and in vitro diagnostics areas. Indeed, there are some interesting parallels between the current state of medical devices, in particular, in vitro diagnostics, and the evolution of microelectronics. While medical tests have traditionally been completed in central laboratories that are filled with specialized equipment and trained technicians, there is currently a trend to complete more and more tests using portable instrumentation. Therefore, the point-of-care medical device area represents the fastest growing sector of in vitro diagnostics.

Tremendous advances have been made recently in the area of LOC devices exploiting the advantages of miniaturization mediated by the small reagent and sample volumes required. Smaller sample and reagent volumes translate to rapid analysis times and less waste volumes, and result in more cost-effective assays that can be operated with less technological constraints making them suitable as a high throughput biomarker validation tool and amenable to point-of-care testing (POCT) (Tudos et al., 2001). Most importantly, these characteristics, when fully developed into a functional system, have the potential to lead to a significant reduction in the time that is needed for an accurate biomarker testing for the diagnosis and subsequent treatment of heart disease.

The tools of the nano materials and microelectronics have been combined and adapted for the practical implementation of miniaturized sensors that are suitable for a variety of important applications. The performance metrics of these miniaturized sensor systems have been shown to correlate closely with established macroscopic gold standard methods, making them suitable for use as subcomponents of highly functional detection systems for analysis of complex fluid samples. These efforts remain unique in terms of functional LOC methods having a demonstrated capacity to meet or exceed the analytical characteristics (sensitivity, selectivity, assay variance, limit of detection) of mature macroscopic instrumentation for a variety of analyte systems including: pH, DNA oligonucleotides, metal cations, biological co-factors, and inflammation markers in serum and saliva (Christodoulides et al., 2002; Curey et al., 2001; Goodey et al., 2001; Goodey and McDevitt, 2003; Lavigne et al., 1998; McCleskey et al., 2003a; McCleskey et al., 2003b; Wiskur et al., 2003; Ali et al, 2003; Rodriguez et al., 2005; Christodoulides et al., 2005a; Floriano et al., 2005; Li et al., 2005a; Christodoulides et al, 2005b; Li et al., 2005b).

Having demonstrated the functionality of the subcomponent systems for miniaturized sensor systems, it becomes important now to search for effective strategies that would enable the translation of such promising miniaturized sensor concepts into important clinical applications. Only with the early implementation of the mini-assay systems for real-world clinical testing will the modular assay system be developed in a manner that will service the future needs of clinicians and the research communities. While the ultimate goal of such research endeavors is to develop universal assay systems that can be reprogrammed rapidly for new application, the steps taken here will target the development of a multi-analyte-based screening that can support clinical research and clinical treatment of patients with heart disease, especially, AMI.

In certain embodiments, the present invention address the need for multiplexed, multi-class LOC assays for a more efficient screening, classification and staging of AMI risk in serum and/or saliva. The LOC sensor array platform could perform chemical and immunological reactions on and/or within the interior regions of microspheres positioned in the inverted pyramidal microchamber wells of a silicon or plastic microchip. For example, microfluidic structures deliver a series of small-volume reagents and washes to the chip and to each of the microspheres. Optical signals generated by the reactions on the microspheres may be visualized at, and captured by, a charge-coupled device (CCD) video chip along with the use of transfer optics. Using the LOC system, complex immunological assays can be performed with small sample volumes, short analysis times, and markedly reduced reagent costs. This integrated and automated system has been developed for the measurement of multiple cardiac biomarkers in the context of serum and saliva measurements. This newly fashioned ultra-sensitive method extends the saliva-based diagnostics to significantly lower analyte levels, as needed for measurement of multiple analytes in patients with AMI.

As a clinical research tool, the LOC device offers the ability to perform multiplex assays in small sample volumes. Additionally, the versatility of this system and its demonstrated enhanced sensitivity makes it a sensitive biomarker quantification tool, while at the same time amenable to applications involving a variety of bodily fluids, such as saliva, in which the analyte concentration may be extremely low (Goodey et al., 2001; Christodoulides et al., 2005b). For example, salivary biomarkers that were previously undetectable by standard methods, may now be targeted with the LOC device to assess systemic disease in a non-invasive fashion (Christodoulides et al., 2005b).

B. Immunoassay Test

In many common diagnostic tests, antibodies may be used to generate an antigen specific response. Techniques for producing an immune response to antigens in animals are well known.

An antibody may be coupled to a polymeric bead. The antibody may then act as a receptor for the antigen that was introduced into the animal. In this way, a variety of chemically specific receptors may be produced and used for the formation of a chemically sensitive particle. Once coupled to a particle, a number of well-known techniques may be used for the determination of the presence of the antigen in a fluid sample. These techniques include radioimmunoassay (RIA), microparticle capture enzyme immunoassay (MEIA), fluorescence polarization immunoassay (FPIA), and enzyme immunoassays such as enzyme-linked immunosorbent assay (ELISA). Immunoassay tests, as used herein, are tests that involve the coupling of an antibody to a polymeric bead for the detection of an analyte.

ELISA, FPIA and MEIA tests may typically involve the adsorption of an antibody onto a solid support. The antigen may be introduced and allowed to interact with the antibody. After the interaction is completed, a chromogenic signal generating process may be performed which creates an optically detectable signal if the antigen is present. Alternatively, the antigen may be bound to a solid support and a signal is generated if the antibody is present. Immunoassay techniques have been previously described, and are also described in the following U.S. Pat. Nos. 3,843,696; 3,876,504; 3,709,868; 3,856,469; 4,902,630; 4,567,149 and 5,681,754, all of which are incorporated by reference.

In ELISA testing, an antibody may be adsorbed onto a polymeric bead. The antigen may be introduced to the assay and allowed to interact with an antibody for a period of hours or days. After the interaction is complete, the assay may be treated with a dye or stain, which reacts with the antibody. The excess dye may be removed through washing and transferring of material. The detection limit and range for this assay may be dependent on the technique of the operator.

Microparticle capture enzyme immunoassay (MEIA) may be used for the detection of high molecular mass and low concentration analytes. The MEIA system is based on increased reaction rate brought about with the use of very small particles (e.g., 0.47 μm in diameter) as the solid phase. Efficient separation of bound from unbound material may be captured by microparticles in a glass-fiber matrix. Detection limits using this type of assay are typically 50 ng/mL.

Recent developments in particle array technology have made it possible to perform immunoassays using microspheres (microbeads). The best-established microsphere assay system is the xMap system (Luminex Corp., Austin, Tex.), which incorporates three well-developed technologies: bioassays, solution-phase microspheres, and flow cytometry. The microsphere assay technology developed by Luminex is ideally suited to a wide range of applications in diagnostics. Immunoassays based on this particle array technology can overcome the problems associated with the traditional ELISAs. Some of the distinct advantages of a microsphere immunoassay (MIA) over traditional ELISAs include accuracy; high sensitivity, specificity, and reproducibility; high-throughput sample analysis; and multiplexing capability.

Fluorescence polarization immunoassay (FPIA) may be used for the detection of low-molecular mass analytes, such as therapeutic drugs and hormones. In FPIA, the drug molecules from a patient serum and drug tracer molecules, labeled with fluorescein, compete for the limited binding sites of antibody molecules. With low patient drug concentration, the greater number of binding sites may be occupied by the tracer molecules. The reverse situation may apply for high patient drug concentration. The extent of this binding may be measured by fluorescence polarization, governed by the dipolarity and fluorescent capacity.

C. Proteomics/Protein Chips

Proteomics/protein chips, also referred to as protein arrays or protein microarrays, are modeled after DNA microarrays. The success of DNA microarrays in large-scale genomic experiments inspired researchers to develop similar technology to enable large-scale, high-throughput proteomic experiments. Protein chips enable researchers to quickly and easily survey the entire proteome of a cell within an organism.

Today there are many companies manufacturing protein chips using many types of techniques including spotting and gel methods. The types of protein chips available include “lab on a chip”, antibody arrays and antigen arrays, as well as a wide range of chips containing “alternative capture agents” such as proteins, substrates and nucleic acids.

Analysis of protein chips comes with many challenges including dynamic protein concentrations, the sheer number of proteins in a cell's proteome, and the understanding of the probes for each protein. Steps include the reading of the protein levels off the chip, and then the use of computer software to analyze the massive amounts of data collected.

Applications of protein chip experiments in the present invention include identifying AMI biomarkers, investigating protein-protein interactions, and testing for the presence of AMI biomarkers in a sample, thus serving as an alternative or complementary advanced detection methodology in addition to the above methods for AMI diagnostics and risk assessment.

### III. AMI BIOMARKERS

A major limitation of the current biomarker approach is the lack of a common assay platform that allows for a multi-marker testing strategy that scans different analyte classes. Therefore, new methods have been developed by the inventors to offer optimal AMI diagnostics based on information from mult-analyte-based screening based on measurement of unique combinations of biomarkers.

A. Importance of Biomarker-Based Diagnostics

In its initial, but crucial stages, CAD is indeed a silent disease whereby a series of molecular- and cellular-level events occur within the vasculature, long before the obvious clinical manifestations begin to appear. Unfortunately, the occurrence of ACS is most often unpredictable because the underlying events responsible for it frequently occur without any obvious clinical symptoms. In fact, not even coronary angiography, the current gold standard for diagnosis of CAD, is capable of identifying these events as this method only provides a negative image of the internal lumen of a blood vessel and lacks the capability to adequately evaluate the vessel wall where an atherosclerotic plaque actually develops (Nakamura et al., 2004).

Early medical intervention in high-risk individuals is an ideal way to combat ASHD. However, in current medical practice, CAD risk assessment tools fail to detect an alarmingly large number of such individuals that suffer significant pain, lose cardiac function and in some cases die. In many such cases, the adverse outcome can be prevented by early intervention with existing medication. Ultimately, since most of these risk factors are modifiable, their early identification is crucial to the survival of the patient. If a cardiac risk pattern (profile) is identified in a prompt, accurate and efficient way, then a highly specific secondary prevention drug regimen for cardiovascular disease can be applied (aspirin, statins, and beta-blockers and ACE-inhibitor therapies). Such treatments are modifiable on an individual basis as a means to prevent and thus alter the adverse outcome of a first cardiac event.

Although atherosclerosis was formally considered a bland lipid storage disease, major advances in basic, experimental and clinical science over the last decade established its strong association with inflammation. Insights gained from the link between inflammation and atherosclerosis have defined specific protein biomarkers, as well as cells, as independent risk factors for heart disease that can now yield predictive and prognostic information of considerable clinical utility (Libby et al., 2002).

In the last decade, there has been an explosion of scientific (basic and clinical) research that has contributed to an increased understanding of the specific mechanisms and pathological pathways that result in heart attacks (or AMI). Inflammation has been identified as a major contributor to the heart disease process. Further, there have been a large number of important studies that have identified a plethora of relevant biomarkers with potential diagnostic and prognostic utility.

Several factors have converged to enhance interest in biomarkers in contemporary diagnostic cardiovascular medicine. First, considerable advances have been made in the understanding of the patho-physiological processes that contribute to various stages of cardiovascular disease. For example, a significant number of protein biomarkers are identified as contributors to various stages of the cardiac cascade, from plaque formation to myocardial infarction (Vasan, 2006).

Second, clinicians face an ever-increasing array of treatment options for patients with cardiovascular disease, and risk becoming overwhelmed by the number of choices they must make for common disorders. Many clinicians have become frustrated by the “one size fits all” approach advocated by guideline committees and staunch proponents of evidenced-based medicine. By providing a window into underlying patho-physiology, biomarkers offer the potential for guiding a more individualized approach to treatment of cardiovascular disease in the future.

Finally, novel technologies now permit rapid identification and purification of high-affinity monoclonal antibodies against potentially important plasma proteins. High-throughput robotic assay methods have also been developed that allow performance of large-scale screening of stored blood samples in a relatively short period of time. Thus, both clinical demand for newer risk stratification tools and “supply” of novel biomarkers have increased concurrently. From this context, it is important to consider that tools for diagnosis and risk stratification in AMI are evolving in three parallel, and closely-associated, directions aimed for the analysis of circulating protein biomarkers, cell-surface markers and genetic polymorphisms.

B. Biomarkers

In certain embodiments, CRP and one or more biomarkers will be used for diagnosis and prognosis of AMI. Through this invention, advanced detection methodologies, such as the powerful lab-on-a-chip (LOC) methodology/technology (University Of Texas at Austin), cardiac proteomic chip (University Of Texas at Austin), ELISA and Luminex (University of Kentucky at Lexington), are applied in conjunction with a select panel of cardiac analytes (biomarkers) to diagnose accurately and efficiently acute myocardial infraction (AMI). Here, multiplexed assays provide an efficient screening of an important aspect of cardiac disease, such as that of AMI event, using sample such as serum and saliva bodily fluids.

Here, serum biomarkers with utility for AMI diagnosis include, but are not limited to: Cardiac troponin I (cTnI), C-reactive protein (CRP), β-natriuretic peptide (BNP), creatine kinase-MB (CK-MB), myeloperoxidase (MPO), matrix metalloproteinase-9 (MMP-9), E-Selectin and interleukin-6 (IL-6).

Here, saliva biomarkers with utility for AMI diagnosis include, but are not limited to: CRP, IL-1β, soluble Vascular Cellular Adhesion Molecule-1 (sVCAM-1), fractalkine, soluble Intercellular Adhesion Molecule-1 (sICAM-1), MMP-9, IL-6 and cTnI.

In general, AMI biomarker analytes that can be quickly assayed to determine whether a patient is at risk of an eventual AMI include platelet activation markers, pro-coagulation markers, inflammatory markers, and cardiac markers. Platelet activation markers include, for instance, platelet membrane P-selectin (mP-selectin), Glycoprotein IIb/IIIa (GPllb/Illa), soluble P-selectin (sP-selectin), and soluble CD40 Ligand (sCD40L). Pro-coagulation markers include, for instance, Prothrombin fragment 1.2 (PTF1.2), D-dimer, and Thrombin Antithrombin III Binding (TAT).

Inflammatory markers include, for example, C-Reactive Protein (CRP), Interleukin-6 (IL-6), intracellular adhesion molecules (e.g., ICAM-1, VCAM-1), matrix metalloproteinases (MMPs, e.g., MMP-1, -2, -3, -4, -5, -6, -7, -9, -10, -11, -12), von Willebrand Factor (vWF), E-selectin and myeoloperoxidase (MPO).

Cardiac markers include Troponin I (Tnl), creatine kinase-MB (CKMB), Myoglobin, fractalkine (CX3CL1) and its receptor, CX3CR1.

Specialty markers include Brain Natriuretic Peptide (BNP), beta-thromboglobulin (BTG), platelet factor 4 (PF4), platelet/endothelial cell adhesion molecule 1 (PECAM-1), soluble fibrin, glycogen phosphorylase-BB, thrombus precursor protein (TPP), Interleukin-1 receptor family/ST2, Interleukin 6 (IL-6), Interleukin 18 (IL-18), placental growth factor (PIGF), pregnancy-associated plasma protein A (PAPP-A), glutathione peroxidase, plasma thioredoxin, Cystatin C, serum deoxyribonuclease I, heart type fatty acid binding protein (H-FABP), and ATP/ADP.

One such biomarker now contributing in a significant manner to the understanding and diagnosis of AMI is C-reactive protein (CRP). The biomarker CRP was originally identified as a substance observed in the plasma of patients with acute infections that reacted with the pneumococcal C-polysaccharide. It is now classified as a characteristic acute phase reactant in human serum and a classic marker of inflammation (Kushner and Rzewnicki, 1994). This important inflammation marker is derived from the liver and interestingly, according to recent studies, from vascular endothelial cells (Venugopal et al., 2005).

C-reactive protein (CRP) is a sensitive but non-specific marker for inflammation. Elevated CRP levels, especially measured with high sensitivity assays, can predict the risk of AMI, as well as stroke and development of diabetes. However, due to its nonspecificty, the use of high sensitivity CRP assays as a means of screening the general population is advised against, but it may be used optionally at the physician's discretion, in patients who already present with other risk factors or known coronary artery disease. Whether CRP plays a direct role in atherosclerosis remains uncertain.

CRP production is regulated by cytokines, such as TNFα, IL-1β and IL-6. The biomarker IL-6, as the major initiator of the acute phase response, induces the synthesis of CRP, as well as that of other acute phase reactants (Baumann and Gauldie, 1990; Baumann et al., 1990; Depraetere et al., 1991; Ganaphthi et al., 1991; Ganter et al., 1989; Toniatti et al., 1990). Given the role of IL-6 in CRP regulation, the combined use of IL-6 and CRP protein levels as indicators of inflammation may provide a better prediction of risk associated with inflammation than would use of either indicator alone (Harris et al., 1999).

Interestingly, when biomarkers TnI, BNP, and CRP are used together, they enhance risk stratification compared with the use of these markers individually (Sabatine et al., 2002). These important studies demonstrate that a simple integer score in which 3 distinct biomarkers are evaluated provide excellent risk stratification in CAD.

Cardiac biomarkers hold great promise as tools to better understand individual differences in the pathobiology of AMI, and may ultimately help individualize treatment strategies (Ridker et al., 2005). For example, in patients with ACS, creatinine kinase-MB and troponins have been firmly established as cardiac biomarkers of myocardial necrosis, which not only assist in the diagnosis of myocardial infarction (MI), but also help to direct treatment (Morrow et al., 2001). BNP serves as a marker of hemodynamic stress and neurohormonal activation in patients with acute and chronic CAD. The same biomarker is strongly associated with the development of death and heart failure, independent of clinical variables and levels of other biomarkers (de Lemos et al., 2001; Kragelund et al., 2005).

In heart failure, BNP and NT-proBNP, have been widely adopted as tools to facilitate heart failure diagnosis and risk stratification (de Lemos et al., 2003; Maisel et al., 2002). Indeed, BNP and NT-proBNP provide more powerful prediction of future risk than any other clinical or biomarker variables identified to date, with risk ratios for death of 3-4 associated with BNP elevation. BNP may help guide medical therapy based on outpatient monitoring. In addition, measurement of NT-proBNP in the Dallas Heart Study (DHS) showed that higher coronary artery calcium scores were independently associated with higher log NT-proBNP levels (p=0.03).

Recently, the potential additional value of troponins has been explored in patients with heart failure. As many as 50% of patients with decompensated heart failure will have evidence of troponin elevation at the time of presentation, and persistent elevation is identified in ˜20-25%. Troponin elevation is associated with excess risk for mortality, and provides incremental and additive prognostic information to BNP (Horwich et al., 2003). However, no single marker or combination of markers exists to adequately predict which patients will develop clinically significant HF or will progress to class IV HF with possible need for mechanical support or cardiac transplantation.

The presence of factors that reflect enhanced thrombogenic activity have also been shown to be associated with an increased risk of recurrent coronary events during long term follow up of patients who have recovered from myocardial infarction. Here, high levels of D-dimer (hazard ratio 2.43; 95% CI, 1.49 to 3.97) and apoB (hazard ratio 1.82; 95% CI, 1.10 to 3.00) and low levels of apoA-I (hazard ratio 1.84; 95%, 1.10 to 3.08) were independently associated with recurrent coronary events, indicating that a procoagulate and a disordered lipid transport contribute independently to recurrent coronary events in post-infarction patients. Most importantly, the risk associated with the combination of all 3 risk factors was multiplicative.

### V. USE OF SALIVA AS A DIAGNOSTIC FLUID

Interest in saliva as a diagnostic medium has increased dramatically during the last decade, as saliva and other oral fluids have been shown to reflect tissue fluid levels of therapeutic, hormonal, immunological, and toxicological molecules. Oral fluids have also been shown to contain bio-markers associated with infectious and neoplastic diseases (Hodinka et al., 1998; Haeckel, 1989; Mandel, 1990; Mandel, 1993a; Schramm et al., 1992). Similarly, the analysis of salivary fluids, like blood-based assays, has the potential to yield useful diagnostic information for the assessment and monitoring of systemic health and disease states, exposure to environmental, occupational, and abusive substances, as well as for the early identification of harmful agents dispersed by bio-terrorist activities (Aguirre et al., 1993).

The major advantages for using saliva in diagnosis relative to blood-based assays have been described in some detail previously (Mandel, 1990; Ferguson, 1987; Mandel, 1993b; Mandel, 1993c; Malamud, 1992; Slavkin, 1998). Saliva collection may be done by procedures that are considered to be non-invasive, painless and convenient. Consequently, these methods may be performed several times a day under circumstances where it may be difficult to collect whole blood specimens.

Many important biological substances including electrolytes (Aps and Martens, 2005; Haeckel and Hanecke, 1993), drugs (Cone, 1993; Jarvis et al., 2000; Svojanovsky et al., 1999; Toennes et al., 2005; Walsh et al., 2003; Zevin et al., 2000), proteins (e.g., cytokines, hormones, enzymes) (Grisius et al., 1997; Hanemaaijer et al., 1998; Lamster et al., 2003; Mogi et al., 1993; Rhodus et al., 2005; Yang et al., 2005), antibodies (Chia et al., 2000; Nogueira et al., 2005; Stroehle et al., 2005), microbes (Stroehle et al., 2005; Lins et al., 2005; Suzuki et al., 2005), and RNAs (Fox et al., 1998; Li et al., 2004a; Li et al., 2004b; St John et al., 2004) have been identified in saliva. Oral fluid presents itself as the ideal diagnostic fluid. There is accumulating evidence that saliva is the “mirror of body”, this makes it a perfect medium to be explored for a non-invasive health and disease monitoring. The translational applications and opportunities are of great potential significance. The ability to classify risk, stratify and monitor health status, disease onset and progression, and treatment outcome monitoring through non-invasive means is a most desirable goal.

In the last decade, saliva has been advocated as a non-invasive alternative to blood as a diagnostic fluid; however, use of saliva has been hindered by the inadequate sensitivity of current methods to detect the lower salivary concentrations of many constituents compared to serum. Furthermore, developments in the areas related to systems for saliva-based point of care diagnostics are complicated by the high viscosity and heterogeneous properties associated with this diagnostic fluid. In certain aspects of the present invention, miniaturized devices and non-invasive sampling procedures that reduce iatrogenic blood loss and pain, present an ideal combination for point-of-care-testing for intensive care situations as applied to AMI diagnosis testing through saliva.

A. Association Between Oral Disease and Cardiovascular Disease

Historically periodontitis has been considered a disease with ramifications localized to the oral cavity, and in much of the population is viewed as a cosmetic problem, with a permanent solution affected by removal of the teeth, i.e. edentulism. However, recent data support that this chronic infection with continued stimulation of the inflammatory responses of the host communicates with the systemic circulation and may contribute to systemic disease sequelae, such as cardiovascular disease (CVD). Indeed, numerous case control and cohort studies have indicated that patients with periodontitis have an increased risk of CVD, e.g., acute myocardial infarction (AMI), when compared with subjects with a healthy periodontium.

However, because evidence of the link has come to light only recently, few studies have looked directly at the mechanisms by which periodontitis might contribute to cardiovascular disease. One possibility is that bacteria from the mouth—or products released by these bacteria—travel through the bloodstream to other parts of the body, where they damage the linings of blood vessels. On the one hand, the association between periodontitis and CVD may be linked through common risk factors such as smoking, diabetes mellitus, aging, male gender, and social-economic factors. On the other hand, there is evidence of periodontitis serving an independent risk factor of CVD (DeStefano et al., 1993; Desvarieux et al., 2005; Joshipura et al., 1996; Mattila et al., 1989). Disturbances in the plasma lipoprotein metabolism, systemic inflammatory reactions as well as local inflammation of the artery wall are considered to contribute to the development of early atherosclerotic lesions in CVD (Blake et al., 2003; Ross, 1999).

Recently, it has been shown that periodontitis is often associated with endotoxemia and mild systemic inflammatory reactions, such as an increase in CRP and other acute phase reactants, while periodontal pathogens have been identified in early atherosclerotic lesions (Haraszthy et al., 2000; Noack et al., 2001; Wu et al., 2000). Furthermore, several groups have reported elevated serum CRP levels in periodontitis patients. The extent of increase in serum CRP levels in periodontitis patients correlates significantly with the severity of the disease, even with adjustments for smoking habits, body mass index, triglycerides, and cholesterol levels. Interestingly, there seems to be an indirect association between the occurrence of periodontal conditions and an increased risk for CVD. The positive correlation between CRP and periodontitis may indicate that circulating inflammatory molecules contribute to the pathogenesis of both conditions and studies that determine the level of CRP, and other inflammation markers, in the fluids of the oral cavity could help us better understand the relationship of these two inflammatory diseases (Noack et al., 2001; Loesche, 1994).

B. Utility of Salivary Diagnostics for Systemic Diseases

In the past, only a few studies targeted the use of saliva as a diagnostic fluid for systemic diseases. Impediments to the use of oral fluids have been the relatively low concentration of various important biomolecules in saliva, in comparison to serum or plasma, accompanied by a lack of sufficiently sensitive assays and equipment that could be used in dental healthcare settings (Kaufman and Lamster, 2004). It remained unclear what salivary analyte targets could be useful as adjunctive clinical information for a systemic disease, such as AMI. Clearly, studies have been needed that define these relationships before the diagnostic utility of saliva could be promoted.

Modern analytical technologies are expected to extend vastly the potential diagnostic value of oral fluids. To be useful, salivary biomarkers must be accurate, biologically relevant, discriminatory, and at measurable concentrations. The identification of these biomarkers for cardiovascular disease, especially, AMI, from the array of potential markers, promises to create a quantum leap in cardiac diagnostics.

### VII. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Measurement of AMI Diagnostic Biomarkers in Body Fluid Samples From a Subject

The initial objective was to explore whether serum biomarkers commonly associated with AMI diagnosis can be detected reliably using unstimulated whole saliva (UWS). The inventors first generated a case-control pilot study examining suitability of saliva for AMI testing using measurement of protein expression levels of both standard and novel biomarkers, in healthy and AMI patients, in both serum and saliva samples.

Demographics of the control group, (59 subjects, average 49.3 years, range 37 to 79 years, 34 females and 25 males, 48 Caucasian, 8 African Americans and 3 Hispanics) were similar to those of the AMI group (56 subjects, average 54.8 years, range 29 to 84 years, 36 females and 20 males, 47 Caucasian and 9 African Americans). The AMI cases had eight fewer teeth and slightly poorer oral health (data not shown), and mean body mass indices were identical (i.e., 28.5).

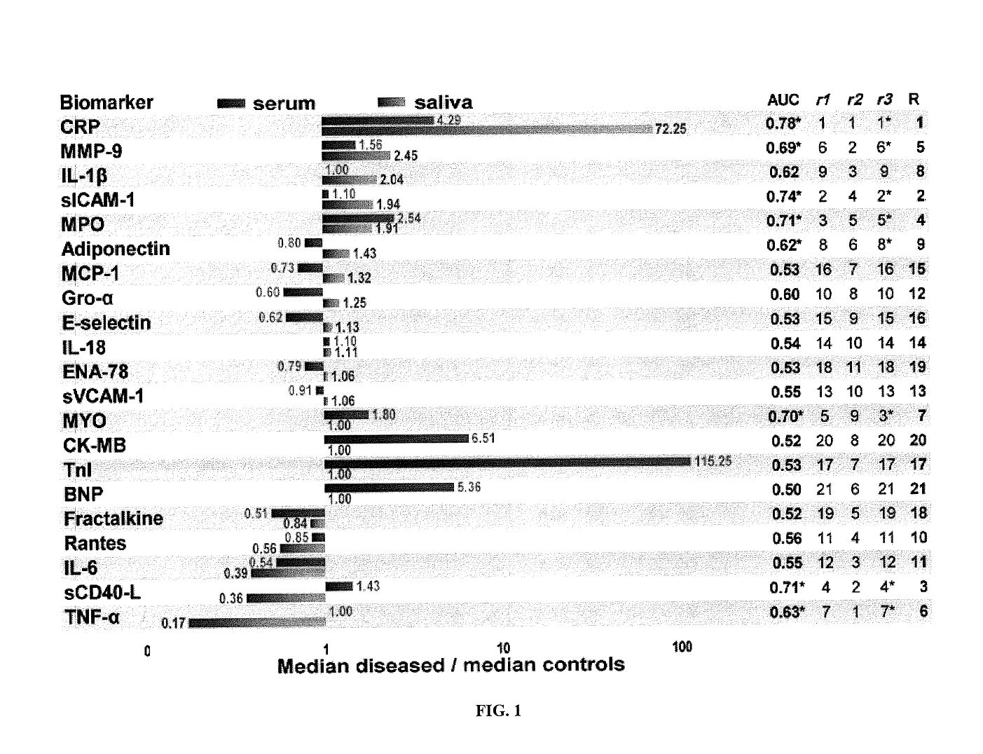

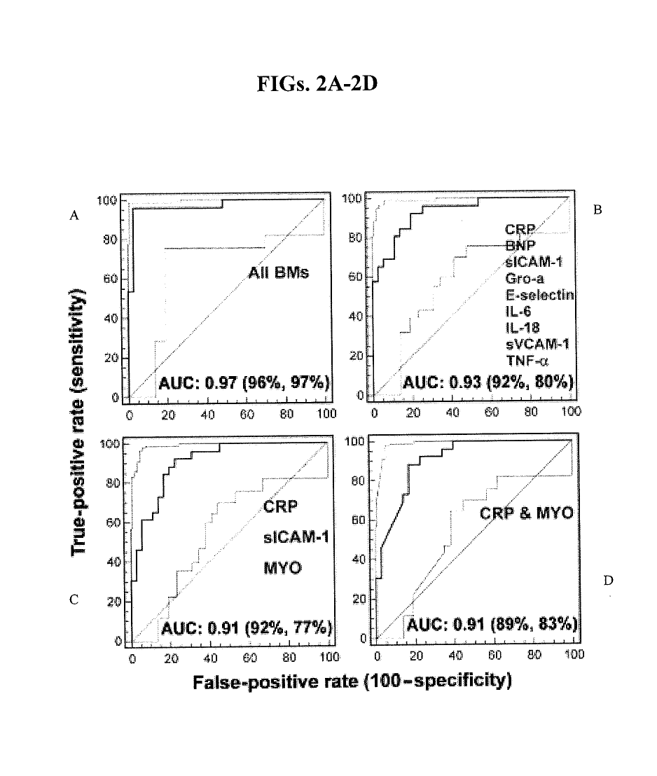

Twenty-one protein biomarkers with relevance to cardiac heart disease patient classification that have a strong literature precedent were studied. In FIG. 1, the greatest ratio in serum protein expression for AMI is seen for cTnI (115), followed by CK-MB (6.5), BNP (5.4), CRP (4.3), MPO (2.5), and MYO (1.8), all exhibiting p<0.0001. Biomarkers MMP-9 (1.6, p=0.004) and sCD40L (1.4, p=0.37), while providing some discriminatory potential yielded more modest ratios. The most down-regulated proteins in serum were fractalkine (0.51, p=0.49), IL-6 (0.54, p=0.030) and Gro-α (0.60, p=0.060). As might be expected, serum-based analysis of combination of established biomarkers yielded strong diagnostic capabilities as demonstrated by AUC values (See FIGS. 1, 2 and Table 1): cTnI (0.99), CK-MB (0.93), BNP (0.90) and MYO (0.77). These values are consistent with previously reported values (6) for same biomarkers of 0.94, 0.91, 0.85, 0.78, respectively, in a study involving more than 2000 patients.

TABLE 1
AUC, SE, P, AUC 95% CI, and best averaged sensitivity and specificity
for various salivary biomarkers and biomarkers combinations.

| Strategy | Biomarker (BM) panel^{a} | AUC | SE | 95% CI | P | Sensitivity | Specificity |
|---|---|---|---|---|---|---|---|
| Aggregate based on R | BM 1 | 0.78 | 0.051 | 0.679-0.865 | 0.0001 | 68.3 | 73.7 |
| Aggregate based on R | BMs 1-2 | 0.77 | 0.052 | 0.668-0.856 | 0.0001 | 68.3 | 76.7 |
| Aggregate based on R | BMs 1-3 | 0.78 | 0.051 | 0.681-0.866 | 0.0001 | 65.1 | 79.1 |
| Aggregate based on R | BMs 1-4 | 0.81 | 0.048 | 0.709-0.887 | 0.0001 | 73.2 | 79.1 |
| Aggregate based on R | BMs 1-5 | 0.81 | 0.048 | 0.705-0.884 | 0.0001 | 73.2 | 79.1 |
| Aggregate based on R | BMs 1-6 | 0.82 | 0.047 | 0.718-0.893 | 0.0001 | 85.4 | 65.1 |
| Aggregate based on R | BMs 1-7 | 0.85 | 0.045 | 0.752-0.920 | 0.0001 | 89.5 | 68.3 |
| Aggregate based on R | BMs 1-8 | 0.85 | 0.044 | 0.752-0.921 | 0.0001 | 89.5 | 70.7 |
| Aggregate based on R | BMs 1-9 | 0.85 | 0.044 | 0.752-0.921 | 0.0001 | 89.5 | 70.7 |
| Aggregate based on R | BMs 1-10 | 0.87 | 0.042 | 0.775-0.935 | 0.0001 | 89.5 | 70.7 |
| All biomarkers | All 21 biomarkers | 0.97 | 0.025 | 0.889-0.996 | 0.0001 | 96.2 | 97.1 |
| BPSFA^{b} | CRP MPO | 0.82 | 0.047 | 0.720-0.895 | 0.0001 | 90.2 | 62.8 |
| BPSFA | CRP MYO | 0.85 | 0.044 | 0.756-0.923 | 0.0001 | 92.1 | 73.2 |
| BPSFA | CRP MPO MYO | 0.85 | 0.045 | 0.746-0.916 | 0.0001 | 92.1 | 68.3 |
| All biomarkers + ECG | All 21 biomarkers + ECG | 1.00 | 0.000 | 0.941-1.000 | 0.0001 | 100.0 | 100.0 |
| BPSFA + ECG | CRP MPO & ECG | 0.95 | 0.026 | 0.872-0.983 | 0.0001 | 90.2 | 90.7 |
| BPSFA + ECG | CRP MYO & ECG | 0.94 | 0.028 | 0.866-0.982 | 0.0001 | 100.0 | 73.2 |
| BPSFA + ECG | CRP MPO MYO & ECG | 0.94 | 0.028 | 0.866-0.982 | 0.0001 | 81.6 | 92.7 |
| Reduced training set | CRP MYO | 0.88 | 0.048 | 0.766-0.953 | 0.0001 | 96.3 | 71.4 |
| Reduced training set | CRP MYO & ECG | 0.96 | 0.028 | 0.869-0.993 | 0.0001 | 92.6 | 85.7 |
| Testing set | CRP MYO | 0.85 | 0.084 | 0.641-0.958 | 0.0001 | 90.2 | 69.2 |
| Testing set | CRP MYO & ECG | 0.89 | 0.073 | 0.693-0.977 | 0.0001 | 81.8 | 92.3 |

^{a}BMs are ranked and combinations assembled according to aggregate score (R) listed in FIG. 1: CRP (1), sICAM-1 (2), sCD40L (3), MPO (4), MMP-9 (5), TNF-α (6), MYO (7), IL-1β (8), adiponectin (9), and RANTES (10).
^{b}BPSFA, Bio markers with precendent in serum that are FDA approved.

Interestingly, the gold standard serum markers of cTnI, CK-MB, BNP and MYO when evaluated for expression levels in UWS yield only modest ratios for distinguishing AMIs from controls. Careful analysis of these samples reveals that while these biomarkers are expressed at measurable levels in extreme phenotypes, typical samples for diseased patients fall below the limit of detection for the Beckman Access instrument when measured at initial time interval available for this study (i.e., within 48 hours).

Novel biomarkers were examined in their capacity to serve as alternative biomarkers for AMI screening. In UWS (FIG. 1), CRP (72, p<0.0001) showed highest ratio in median concentration of AMI/control, followed by MMP-9 (2.5, p=0.0029), IL-1β (2.0, p=0.0659), sICAM-1 (1.9, p=0.0001), MPO (1.9, p=0.0008), adiponectin (1.4, p=0.052), MCP-1 (1.3, p=0.66) and Gro-α (1.2, p=0.16). Most down-regulated proteins in whole saliva were TNF-α (0.17, p=0.038), sCD40L (0.36, p=0.0005) and IL-6 (0.39, p=0.40).

Initially, identifying a series of biomarkers that can be measured reliably in saliva enabled the use of logistic regression as a screening tool for determining the most useful multimarker panels for salivary AMI diagnosis. Larger sample sizes are required to validate these initial models. In FIG. 2, a series of 4 ROC curves are presented for AMI patient classification using various logistic regression algorithms. Options studied included all salivary biomarker inputs regardless of statistical or biological significance. This “enter” model appears to yield the most accurate diagnostic capabilities of the studied cases (0.97, 0.89-1.00 95% CI, p=0.0001), but suffers from over-fitting (see below), and the inclusion of numerous biomarkers that are not approved yet for clinical use. The next model includes only variables that were entered sequentially into the model based on their statistical significance as single markers and resulted in the following salivary biomarkers: BNP, CRP, IL-18, sICAM-1, TNF-α, sVCAM-1, E-selectin, Gro-α, IL-6. This “Forward” model yielded (0.91, 0.80-0.97 95% CI, p=0.0001).

Next, all independent variables were first entered into the model and then removed sequentially if not found significant. This “Backward” model lead to the inclusion of the following salivary biomarkers: CRP, sICAM-1, and MYO, yielding (0.93, 0.84-0.98 95% CI, p=0.0001). Finally, all significant biomarkers were entered sequentially and the model was recalculated after exclusion of any variable found non-significant upon inclusion of another independent variable. This “Stepwise” method indicated that salivary CRP and MYO had large effects and yielded (0.91, 0.80-0.97 95% CI, p=0.0001).

Even with the acquisition of robust protein expression levels across 84 patients in this case-control study, potentially other factors including outlier samples, sample stability and measurement inaccuracies can influence the main conclusions related to the utility of the various salivary biomarkers. To acquire a more resilient understanding of the potential diagnostic capabilities of these salivary biomarkers with such influences the composite data was examined from a number of perspectives. Thus, an aggregate ranking system as summarized in the right panel of FIG. 1 was created for each salivary biomarker using the following three factors: (1) AUC obtained for single biomarkers (AUC in FIG. 1), leading to ranking r1, (2) the value of the ratio of median diseased over median healthy (r2 in FIG. 1), (3) the p-value assessing the statistical significance of the difference between the medians of diseased and control populations (r3 in FIG. 1), and the aggregate rankings averaging the prior three factors (R in FIG. 1).

While further confirmation studies are required to refine expectations for the various biomarker combinations arising from these scoring strategies, these data provide valuable initial insight into the utility of these salivary biomarkers for AMI screens and diagnoses. The aggregate score projects the following list of the top 10 biomarkers and is considered to yield the most valuable information for the diagnosis of AMI from a single salivary biomarker perspective:

CRP (#1), sICAM-1 (#2), sCD40L (#3), MPO (#4), MMP-9 (#5), TNF-α (#6), MYO (#7), IL-1β (#8), adiponectin (#9), and RANTES (#10)

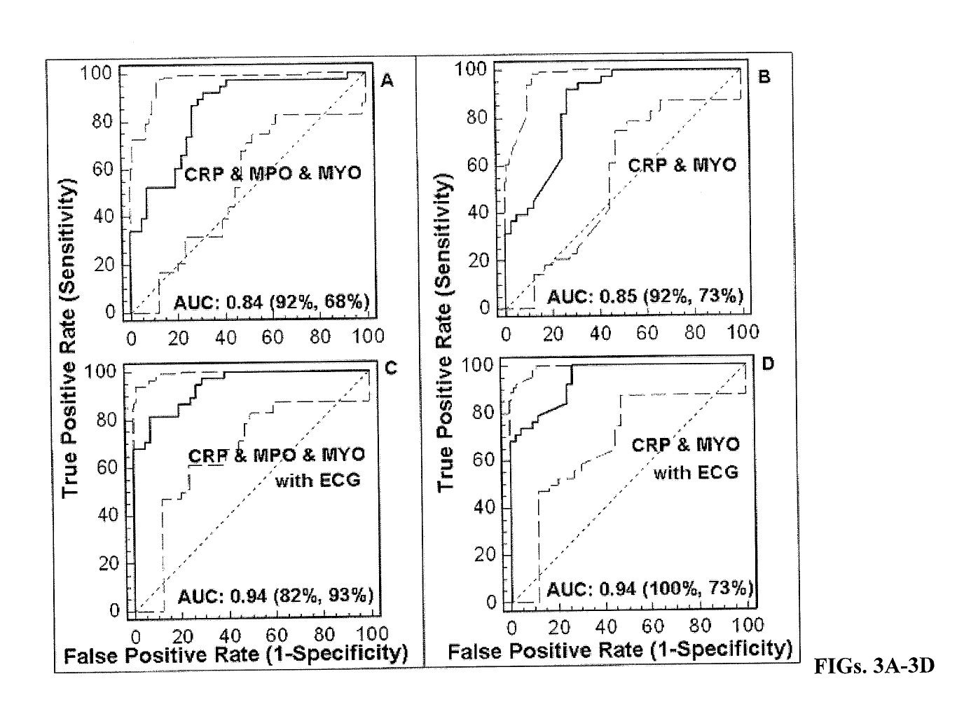

Remarkably, ROC analysis of the binary panels such as salivary CRP-MPO and CRP-MYO, as well as trio panel involving CRP-MPO-MYO (only FDA-approved biomarkers) yielded similar AUC of 0.82, and 0.85, and 0.84, respectively. The ROC curves obtained from the analysis of the selected salivary CRP-MYO-MPO and CRP-MYO panels are shown in FIGS. 3A & 3B. It appears that salivary CRP-MYO serves as the minimal reliable panel that can be assembled from the initial 21 biomarkers. Both of these biomarkers have been approved by the FDA for clinical use, an important consideration for the intended application, although for other indications than saliva AMI screens.

A number of combinations, including up- and down-regulated biomarkers can discriminate with statistical significance between the AMI and control groups, but panels with only 2 or 3 select biomarkers are often found to perform as well or better than more inclusive panels.

The inventors explored the utility of a companion test to ECG, by focusing on using a saliva test to capture those NSTEMI patients that are not diagnosed in the initial ECG screen, 39% of the AMI patients in our case-control study. A new set of ROC curves were built, based on the use of the same panels as previously described, except for the salivary biochemistry data is combined with the ECG screening information (i.e., a value of 1 is input for STEMI patients, while 0 is used for NSTEMI patients) using the latter as an independent variable.

The inclusion of ECG to the panels increased the AUC in most combinations of biomarkers. A focus on two cases with minimal numbers of biomarkers is chosen so as to demonstrate the utility of the combination ECG-salivary test. The established panel of CRP-MPO-MYO in conjunction with ECG yields an AUC of 0.94 (0.87-0.98 95% CI, p=0.0001) when subjected to logistic regression and ROC analysis (FIG. 3C). This same panel demonstrated discrimination between healthy and cardiac disease with 82% sensitivity and 90% specificity. Further, the CRP-MYO panel, while providing a similar AUC displayed 100% sensitivity and 73% specificity (FIG. 3D). These values exceed the capacity of the ECG by itself with sensitivity of only 61% as measured in this study.

This limited dataset was split into training and testing to find in a preliminary manner the ruggedness of the procedure for selecting classification models. Logistic regression coefficients were recalculated and models were established from the data obtained from 55 patients (−⅔ of the total sample population) used in the training set. The CRP and MYO model was challenged with the remainder ⅓ of the samples as external data, alone and as a complement to ECG.

The CRP-MYO biomarker panel yielded similar AUC for the full training set (0.85) and the reduced training set (0.88), with similar sensitivity and specificity values. In addition, consistent increases in AUC were observed for this panel when used in conjunction with ECG (AUC=0.96, 93% sensitivity, 86% specificity). The logit equation obtained from the logistic regression analysis is provided below:

logit(probabililty of AMI)=0.0004365*CRP+0.00278*MYO−2.8253

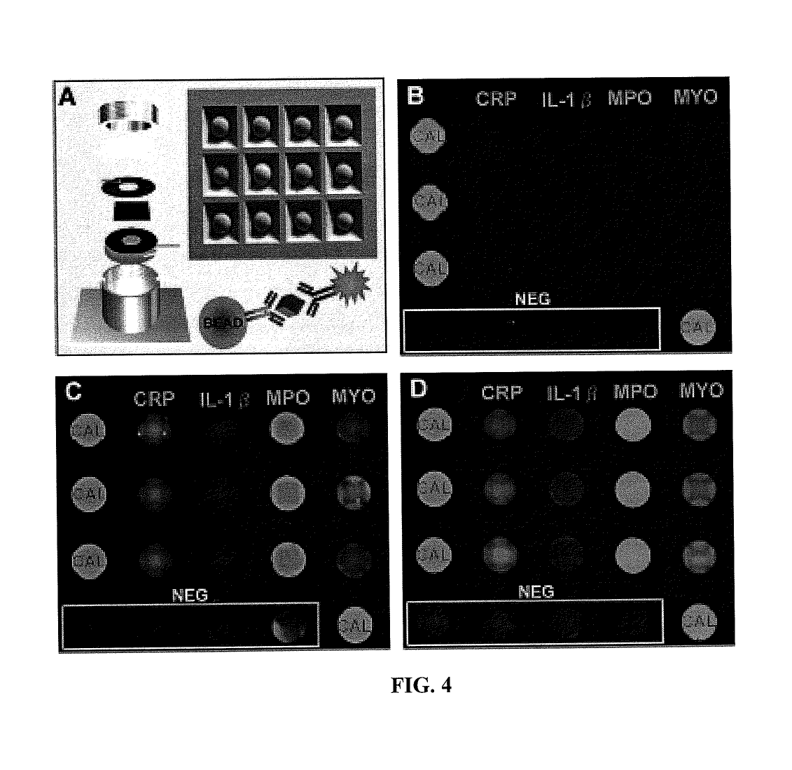

Finally, the translation of a saliva assay to an embodiment that is compatible with POC usage was performed. Multiplexed salivary tests relevant to AMI screen were completed using an LOC developed by the inventors. FIG. 4 shows detection of CRP, MYO, IL-1β, and MPO in fluorescent multiplex assays performed on both AMI and control patients. Just as in the initial biomarker validation phases, the LOC studies also document measurable signal differences in protein fingerprint patterns of these two patient groups. Bead-based immuno-assay systems display strong analytical performance characteristics (typical intra-assay variance of 4-8% and inter-assay variance of 6-10%) (Christodoulides et al., 2005a; Christodoulides et al., 2005b). Correlation studies completed with FDA approved instruments for serum CRP yield R2 values of 0.98. The LOC assay range for CRP, MYO, IL-1β, and MPO all span the physiological ranges of these biomarkers in saliva. Collectively, these results suggest the mini-test system exhibits overall performance characteristics suitable for use in clinical settings.

### Materials and Methods

Study Design, Patient Recruitment and Sample Collection. A cross-sectional clinical case-control study was implemented and 56 subjects recruited within 48 hours of onset of symptoms of AMI, with 59 age- and gender-matched healthy controls at the hospitals of University of Kentucky (UK) and University of Louisville (UL). Recruitment was coordinated with the cardiac care team balancing needs of patients including pain management, reperfusion and family support issues. All subjects were at least 18 years of age. Exclusion criteria were: fever, stroke, immune disorders, steroidal medications use, organ complications/failure, and inability to provide saliva. Rights of all subjects were protected by participating sites Institutional Review Boards. Informed consent was granted prior to sample collection and samples tested were de-identified to ensure privacy rights.

Demographic information was obtained, medical records were reviewed, oral evaluation performed and biological fluids obtained (blood and unstimulated whole saliva) from each subject. Samples were transported to local laboratory on ice, centrifuged, distributed into aliquots and stored at −80° C. until analyzed. Samples from UL were shipped on dry ice to the UK laboratories on a bimonthly basis. Samples were analyzed in duplicate for lipids, cardiac enzymes and a panel of 21 biomarkers using Luminex, ELISA, or Beckman Access in the CLIA-certified UK hospital Clinical Chemistry Laboratory within 3 months of storage.

Oral health was assessed visually using a portable light at bedside following AMI or in dental operatory for control subjects. Oral health was scored as poor, fair or good based on presence or absence of dental complaints, degree of mucosal inflammation, extent of visible decay and periodontal disease.

Measurement of Biomarkers by Luminex and Beckman Access. In this study, standard cardiac biomarkers BNP, MYO, CK-MB and cTnI were measured using Beckman Access. Luminex IS-100 instrument (Luminex Corp. Austin, Tex.) was used for multiplexed detection of 21 biomarkers, relevant to cardiovascular disease, using kits available from Beadlyte Technology (Millipore): C-Reactive Protein (CRP), interleukin-6 (IL-6), monocyte chemoattractant protein-1 (MCP-1), interleukin-1β (IL-1β), myeloperoxidase (MPO), soluble cluster of differentiation ligand (sCD40L), tumor necrosis factor (TNF-α), RANTES, Fractalkine, soluble vascularization cellular adhesion molecule (sVCAM-1), epithelial cell-derived neutrophil-activating peptide 78 (ENA-78), interleukin-18 (IL-18), E-selectin, growth related protein (Gro-α), adiponectin, soluble intra cellular adhesion molecule-1 (sICAM-1), and matrix metallo-proteinase-9 (MMP-9).

Data Analysis. Data mining steps were completed by consolidating information into homogenous datasets to maximize the number of patients for which a complete biomarker panel was available. The process resulted in an 88-patient dataset for serum, composed of 42 controls, 23 NSTEMI and 23 STEMI patients, along with an 84-patient dataset for saliva (43 Controls, 16 NSTEMI, and 25 STEMI patients) from the initial 115 patients. Inability to collect sufficient sample volume for numerous elements of this study was responsible for loss of some of patients. Also, some samples were required for development, validation and testing of the LOC system.

Non-parametric Wilcoxon-Mann-Whitney tests were used to evaluate differences between median biomarkers concentrations detected in saliva of healthy subjects and AMI patients. Medcalc V. 9.5.2.0 (Mariakerke, Belgium) software was used for logistic regression and receiver operating characteristics (ROC) analysis. The ROC curves were constructed and values of the area under the curve (AUC) computed, either from single biomarker concentrations or from predicted values computed through the logistic regression for multimarker panels. Standard error (SE) and two-tailed p-value at the 95% confidence level were determined using these methods.

LOC Multiplexed Test. Design, fabrication and testing methods for LOC structures have been described in detail in previous reports (Goodey et al., 2001; Christodoulides et al., 2002; Christodoulides et al., 2005a; Christodoulides et al., 2005b).

### REFERENCES

The following references, to the extent that they provide exemplary procedural or other details supplementary to those set forth herein, are specifically incorporated herein by reference.
U.S. Patent Appln. 2002 0138208 A1
U.S. Patent Appln. 2002 0193950
U.S. Patent Appln. 2003 0004402
U.S. Patent Appln. 2003 0055615 A1
Aguirre et al., Crit. Rev. Oral Biol. Med., 4(3-4):343-350, 1993.
Ali et al., Analytical Chem., 75(18):4732-4739, 2003.
American Heart Association, Heart and Disease Statistics—2004.
Anderson and Anderson, Molecul. Cell. Proteomics, 1(11):845-867, 2002.
Anderson, J. Physiology-London, 563:23-60, 2005b.
Anderson, Molecul. Cell. Proteomics, 4(10):1441-1444, 2005a.
Aps and Martens, Forensic Sci. Int., 150(2-3):119-31, 2005.
Arab et al., J. Amer. Coll. Cardiol., 48(9):1733-1741, 2006.
Baumann et al., J. Biol. Chem., 265(36):22275-22281, 1990.
Baumann et al., Molec. Biol. Med., 7(2):147-159, 1990.
Bernard et al., Lab. chip, 5:261-269, 2005b.
Blake et al., Europ. Heart J., 24:116-116, 2003.
Chia et al., Oral Microbiol. Immunol., 15(2):131-8, 2000.
Christodoulides et al., Analytical Chem., 74(13):3030-3036, 2002.
Christodoulides et al., Clinical Chem., 51(12):2391-2395, 2005a.
Christodoulides et al., Lab. Chip, 5(3):261-9, 2005b.
Cone, Ann. NY Acad. Sci., 694:91-127, 1993.
Curey et al., Analy. Biochem., 293(2):178-184, 2001.
Curtis et al., Trends in Biotechnology, 23(8):429-435, 2005.
de Lemos et al., Lancet., 362(9380):316-322, 2003.
de Lemos et al., NE J. Med., 345(14):1014-1021, 2001.
Depraetere et al., Agents and Actions, 34(3-4):369-375, 1991.
DeStefano et al., BMJ, 306(6879):688-91, 1993.
Desvarieux et al., Circulation, 111(5):576-82, 2005.
Do et al., Molecules and Cells, 22(3): 254-261, 2006.
Donahue et al., Amer. Heart J., 152(3):478-485, 2006.
Ferguson, J. Dental Res., 66(2):420-424, 1987.
Floriano et al., Biosens. Bioelect., 20(10):2079-2088, 2005.
Fox et al., Adv. Exp. Med. Biol., 438:909-15, 1998.
Fu and Van Eyk, Expert Rev. Proteom., 3(2):237-249, 2006.
Fung et al., Expert Rev. Proteom., 2(6):847-862, 2005.
Ganaphthi et al., J. Immunol., 147(4):1261-1265, 1991.
Ganter et al., Embo. J., 8(12):3773-3779, 1989.
Goodey et al., J. Amer. Chem. Soc., 123(11):2559-2570, 2001.
Goodey et al., J. Amer. Chem. Soc., 125(10):2870-2871, 2003.
Grisius et al., J. Rheumatol., 24(6):1089-1091, 1997.
Haeckel and Hanecke, Ann. Biol. Clin. (Paris), 51(10-11):903-10, 1993.
Haeckel, J. Clinical Chem. Clinical Biochem., 27 (4):223-226, 1989.
Hanemaaijer et al., Matrix Biol., 17(8-9):657-65, 1998.
Haraszthy et al., J. Periodontol., 71(10):1554-60, 2000.
Harris et al., Amer. J. Med., 106(5):506-512, 1999.
Herrmann, Cardiovas. Res., 60(2):220-222, 2003.
Hodinka et al., Clin. Diag. Lab. Immun., 5(4):419-426, 1998.
Hortin et al., Clinical Chem., 52:1218-1222, 2006.
Horwich et al., Circulation, 108(7):833-838, 2003.
Huang, J. Immunol. Methods, 255(1-2):1-13, 2001.
Jain, “Statistical Pattern Recognition: A Review”, IEEE Transactions on Pattern Analysis and Machine Intelligence, Vol. 22, No. 1, January 2000
Jarvis et al., BMJ, 321(7257):343-345, 2000.
Joshipura et al., J. Dent. Res., 75(9):1631-6, 1996.
Jung et al., Biometrical J., 48(2):245-254, 2006.
Kaufman and Lamster, J. Clin. Periodontol., 27(7):453-65, 2000.
Kragelund et al., NE J. Med., 352(7):666-675, 2005.
Kushner and Rzewnicki, Baillieres Clinical Rheumat., 8(3):513-530, 1994.
Lam et al., J. Cardiology, 108(1):12-19, 2006.
Lamster et al., J. Periodontol., 74(3):353-9, 2003.
Lavigne et al., J. Amer. Chem. Soc., 120(25):6429-6430, 1998.
Lee et al., Crit. Rev. Clinical Lab. Sci., 44(1):87-114, 2007.
Li et al., Biosens. Bioelect., 21:574-580, 2005a.
Li et al., Clin. Cancer Res., 10(24):8442-50, 2004a.
Li et al., J. Dent. Res., 83(3):199-203, 2004b.
Li et al., J. Microelectromech., Sys., 14(6), 2005b.
Libby et al., Circulation, 105(9):1135-1143, 2002.
Lins et al., J. Med. Virol., 77(2):216-20, 2005.
Liszewski, Genetic Engineering News, 26(6):1-+, 2006.
Liu et al., Analytical Chem., 75(18):4718-4723, 2003.
Loesche, Compendium, 15(8):976, 978-82, 985-6, 1994.
Ludwig and Weinstein, Nature Rev. Cancer, 5(11):845-856, 2005.
Maisel et al., NE J. Med., 347(3):161-167, 2002.
Malamud, Br. J. Med., 305(6847):207-208, 1992.
Mandel, Annals NY Acad. Sciences., 694:1-10, 1993a.
Mandel, Crit. Rev. Oral Biol. Med., 4(3-4):599-604, 1993c.
Mandel, J. Amer. Dental Assoc., 124(1):85-87, 1993b.
Mandel, J. Oral Path. Med., 19(3):119-125, 1990.
Manz et al., Sensors Actuators B-Chemical, 1(1-6):244-248, 1990.
Mattila et al., BMJ, 298(6676):779-81, 1989.
Mayr et al., Molecul. Cell. Proteomics, 5(10):1853-1864, 2006.
McCleskey et al., Angewandte Chemie-International Ed., 42(18):2070-2072, 2003b.
McCleskey et al., J. Amer. Chem. Soc., 125(5):1114-1115, 2003a.
Michel et al., J. Cardio-Thoracic Surg., 23:684-687, 2003.
Mogi et al., Arch. Oral Biol., 38(12):1135-9, 1993.
Morrow et al., Jama-J. Amer. Med. Assoc., 286(19):2405-2412, 2001.
Nakamura et al., Rev. Cardiovasc. Medicine, Suppl. S22-S33, 2004.
Napoli et al., Heart, 89(6):597-604, 2003.
Noack et al., J. Periodontol., 72(9):1221-1227, 2001.
Nogueira et al., Infect. Immun., 73(9):5675-84, 2005.
Office of Technology Commercialization 27(21) IPQ for The University of Texas at Austin Biological Sciences
Office of Technology Commercialization Page 20 of 27 IPQ for The University of Texas at Austin Biological Sciences
Omenn, Proteomics, 6(20):5662-5673, 2006.
PCT Appln. WO 2000/004372
PCT Appln. WO 2001/031580
PCT Appln. WO 2001/006239
PCT Appln. WO 2001/006244
PCT Appln. WO 2001/006253
PCT Appln. WO 2001/055701
PCT Appln. WO 2001/055702
PCT Appln. WO 2001/055703
PCT Appln. WO 2001/055704
PCT Appln. WO 2001/055952
PCT Appln. WO 2002/061392
PCT Appln. WO 2003/090605
PCT Appln. WO 2004/009840
PCT Appln. WO 2004/072097
PCT Appln. WO 2004/072613
PCT Appln. WO 2005/059551
PCT Appln. WO 2005/083423
PCT Appln. WO 2005/085796
PCT Appln. WO 2005/085854
PCT Appln. WO 2005/085855
PCT Appln. WO 2005/090983
PCT Appln. WO 2007/002480
PCT Appln. WO 2007/005666
PCT Appln. WO 2007/134189
PCT Appln. WO 2007/134191
Quackenbush, Nature Genetics, 32:496-501, 2002.
Rhodus et al., Cancer Detect. Prey., 29(1):42-5, 2005.
Ridker, Amer. Heart J., 148(1):S19-S26, 2004.
Ridker, NE J. Med., 352(1):20-28, 2005.
Ridker et al., NE J. Med., 347:1557-1565, 2002.
Rifai and Ridker, Clin. Chem., 49:666-669, 2003.
Rodriguez et al., Plos. Medicine, 2(7):663-672, 2005.
Ross, NE J. Med., 340(2):115-126, 1999.
Sabatine et al., Circulation, 105(15):1760-1763, 2002.
Schramm et al., J. Analytical Toxicol., 16(1):1-9, 1992.
Situma et al., Analytical Biochem., 340(1):123-135, 2005.
Slavkin, J. Amer. Dental Assoc., 129(8):1138-1143, 1998.
Sluss, Point of Care, 5:38-46, 2006.
St John et al., Arch. Otolaryngol. Head Neck Surg., 130(8):929-35, 2004.
Stephan et al., Prostate, 66(6):651-659, 2006.
Stroehle et al., J. Parasitol., 91(3):5.61-3, 2005.
Suzuki et al., J. Clin. Microbiol., 43(9):4413-7, 2005.
Svojanovsky et al., J. Pharm. Biomed. Anal., 20(3):549-555, 1999.
Toennes et al., Forensic Sci. Int., 152(2-3):149-55, 2005.
Toniatti et al., Molec. Biol. Med., 7(3):199-212, 1990.
Toumpoulis et al., Europ. J. Cardio-Thoracic Surg., 27:128-133, 2005
Tudos et al., Lab. Chip, 1(2):83-95, 2001.
Vasan, Circulation, 113(19):2335-2362, 2006.
Venugopal et al., Amer. J. Pathol., 166(4):1265-1271, 2005.
Verhoeckx et al., Proteomics, 4(4):1014-1028, 2004.
Verpoorte and De Rooij, Proceed. Ieee, 91(6):930-953, 2003.
Walsh et al., J. Anal. Toxicol., 27(7):429-39, 2003.
Whitesides, Small, 1(2):172-179, 2005.
Wu et al., Arch. Intern. Med., 160(18):2749-2755, 2000.
Wu, Point of Care, 5:20-24, 2006.
Yang et al., Lab. Chip., 5(10):1017-23, 2005.
Zevin et al., Drug Alcohol Depend., 60(1):13-8, 2000.
Zhu et al., Genome Biol., 7(11), 2006.
Zolg, Molecul. Cell. Proteomics, 5(10):1720-1726, 2006.

1-5. (canceled)

6. A method for establishing a diagnosis that a subject has suffered from acute myocardial infarction or a prognosis that a subject is at risk of suffering from acute myocardial infarction, the method comprising:
simultaneously measuring a level of two or more biomarkers in a sample from a subject, wherein a first biomarker is C-reactive protein (CRP) and a second biomarker is selected from the group consisting of cardiac troponin I (cTnI), MMP-9, IL-6, IL1β, soluble Vascular Cellular Adhesion Molecule-1 (sVCAM-1), fractalkine, soluble Intercellular Adhesion Molecule-1 (sICAM-1), B-natriuretic peptide (BNP), creatine kinase-MB (CK-MB), myeloperoxidase (MPO) and E-Selectin; and
comparing the level with a reference level; and
establishing the diagnosis or the prognosis of the subject with regard to acute myocardial infarction.

7. The method of claim 6, wherein the sample is a fluid sample.

8. The method of claim 7, wherein the fluid sample is serum.

9. The method of claim 7, wherein the fluid sample is saliva.

10. The method of claim 8, wherein the second biomarker is selected from the group consisting of cardiac troponin I (cTnI), MMP-9, IL-6, B-natriuretic peptide (BNP), creatine kinase-MB (CK-MB), myeloperoxidase. (MPO) and E-Selectin.

11. The method of claim 9, wherein the second biomarker is selected from the group consisting of cardiac troponin I (cTnI), MMP-9, IL-6, IL1β, soluble Vascular Cellular Adhesion Molecule-1 (sVCAM-1), fractalkine and soluble Intercellular Adhesion Molecule-1 (sICAM-1).

12. The method of claim 11, wherein the second biomarker comprises at least two biomarkers.

13. The method of claim 12, wherein the second biomarker comprises two biomarkers.

14. The method of claim 12, wherein the second biomarker comprises three biomarkers.

15. The method of claim 12, wherein the second biomarker comprises four biomarkers.

16. The method of claim 12, wherein the second biomarker comprises five biomarkers.

17. (canceled)

18. The method of claim 12, wherein the second biomarker comprises six biomarkers.

19. The method of claim 12, wherein the second biomarker comprises seven biomarkers.

20. The method of claim 6, wherein the two or more biomarkers comprise at least three biomarkers, and wherein the third biomarker comprises LDL, HDL, adiponectin, Apolipoprotein A (ApoA), Apolipoprotein B (Apo B), IL-1α, IL-4, IL-5, IL-10, IL-13, IL-18, FABP (cardiac fatty acid protein), TNF-α, MCP-1, sCD40L, ENA78, PIGF, PAPP-A, RANTES, sCD40L, von Willebrand Factor (vWF), D-dimer, IMA, FFAu, Choline, cTnT, Myoglobin, NT-proBNP, MMP, or a combination thereof.

21. The method of claim 6, wherein the level is measured by one of a microfluidic sensor array, an immunoassay test, a μ-array measurement, a proteomic array, and a micros here assay system that incorporates bioassays, solution-phase microspheres, and flow cytometry.

22. The method of claim 21, wherein the level is measured by the microfluidic sensor array, and the microfluidic sensor array is a lab-on-a-chip (LOC) sensor.

23. The method of claim 21, wherein the level is measured by the immunoassay test, and wherein the immunoassay test is an enzyme-linked immunosorbent assay (ELISA).

24. The method of claim 6, wherein the reference level is determined by measuring a level of the biomarkers in a population of subjects having no acute myocardial infarction symptoms.

25. The method of claim 6, wherein the acute myocardial infarction is a recurrent cardiac event.

26. The method of claim 10, wherein the second biomarker comprises at leas two biomarkers.

27. The method of claim 26, wherein the second biomarker comprises one of three, four, five, six, and seven biomarkers.

28. A method for evaluating a subject suspected of having suffered an acute myocardial infarction, the method comprising simultaneously measuring levels of C-reactive protein (CRP), myoglobin, and myeloperoxidase (MPO) in a saliva sample obtained from a subject suspected of suffering an acute myocardial infarction.

29. The method of claim 28, further comprising measuring the levels of one or more of cardiac troponin I (cTnI), MMP-9, IL-6, IL1β, soluble Vascular Cellular Adhesion Molecule-1 (sVCAM-1), fractalkine, soluble Intercellular Adhesion Molecule-1 (sICAM-1), B-natriuretic peptide (BNP), creatine kinase-MB (CK-MB), and E-Selectin in the saliva sample.

30. The method of claim 28, wherein the level is measured by a microfluidic sensor array.

31. The method of claim 30, wherein the microfluidic sensor array is a lab-on-a-chip (LOC) sensor.

32. A method for evaluating a subject suspected of having suffered an acute myocardial infarction, the method comprising simultaneously measuring levels of BNP, CRP, IL-18, sICAM-1, TNF-α, sVCAM-1, E-selectin, Gro-α, and IL-6 in a saliva sample obtained from a subject suspected of suffering an acute myocardial infarction.
